(19) United States
(12) Patent Application Publication
(10) Pub. No.: US 2023/0024565 A1
(43) Pub. Date: Jan. 26, 2023

(54) ELECTROSURGICAL INSTRUMENT

(71) Applicant: GYRUS MEDICAL LIMITED, Cardiff (GB)

(72) Inventors: Ben CLARKE, Cardiff (GB); James Alan DICKSON, Cardiff (GB); Thomas LLOYD, Cardiff (GB)

(73) Assignee: GYRUS MEDICAL LIMITED, Cardiff (GB)

(21) Appl. No.: 17/870,179

(22) Filed: Jul. 21, 2022

(30) Foreign Application Priority Data
Jul. 26, 2021 (GB) 2110683.6

(51) Int. Cl. A61B 18/04

(52) U.S. Cl. CPC A61B 18/04; A61B 2018/00714

(57) ABSTRACT

A temperature sensing system for an electrosurgical instrument able to detect temperatures internal and/or external to the electrosurgical instrument. Temperatures detected by a temperature sensor are processed by a monitoring module which prompts action to reduce temperatures where appropriate. The temperature sensing system is particularly useful for electrosurgical instruments which combine rotary shaver arrangements and RF electrode arrangements, where suction is used to remove RF heated saline from the surgical site. Without monitoring the temperature of the electrosurgical instrument and/or the surgical site, there is a risk of burning the patient if the RF heated saline becomes too hot as the electrosurgical instrument may not be adequately insulated.

### TECHNICAL FIELD

Embodiments of the present invention described herein relate to an electrosurgical device, and in particular to a temperature sensing system for an electrosurgical device, the device having shaver, RF and suction capabilities.

### BACKGROUND TO THE INVENTION AND PRIOR ART

Electrosurgical instruments provide advantages over traditional surgical instruments in that they can be used for coagulation and tissue sealing purposes. Surgical apparatus used to shave, cut, resect, abrade and/or remove tissue, bone and/or other bodily materials are known. Such surgical apparatus can include a cutting surface, such as a rotating blade disposed on an elongated inner tube (or shaft) that is rotated within an elongated outer tube (or shaft) having a cutting window. The inner and outer tubes together form a surgical cutting instrument or unit. In general, the elongated outer tube includes a distal end defining an opening or cutting window disposed at a side of the distal end of the outer tube. The cutting window of the outer tube exposes the cutting surface of the inner tube (located at a side of the distal end of the inner tube) to tissue, bone and/or any other bodily materials to be removed. A powered handpiece is used to rotate the inner tube with respect to the outer tube while an outer tube hub (connected to the proximal end of the outer tube) is fixed to the handpiece and an inner tube hub (connected to the proximal end of the inner tube) is loosely held in place by the powered handpiece.

In some instruments the inner tube is hollow and has a cutting window on a side surface of its distal end such that tissue, bone, etc. will be cut or shaved as the cutting window of the inner tube aligns with and then becomes misaligned with the cutting window of the outer tube as the inner tube is rotated within the outer tube. In this regard, it can be said that the cutting device removes small pieces of the bone, tissue, etc. as the inner tube is rotated within the outer tube.

In some instruments a vacuum is applied through the inner tube such that the bodily material that is to be cut, shaved, etc. is drawn into the windows of the inner and outer tubes when those windows become aligned, thereby facilitating the cutting, shaving, etc. of the tissue, which then travels through the inner tube due to the suction. It also is common to supply an irrigation fluid, which can include a liquid, to the surgical site via a passage provided between the inner and outer tubes. Alternatively, saline fluid inflow may be separate and provided by an inflow cannula.

Many times during surgery, the surgeon wishes to apply RF energy to either coagulate bleeding vessels, or ablate tissue in the surgical site without performing cutting with a shaver instrument. This usually is done by withdrawing the shaver instrument and inserting a dedicated RF ablation/coagulation/suction device (for example, a RF wand which is a tube to which suction is applied). However, exchanging the surgical tool for the dedicated RF wand is time-consuming. Furthermore, insertion and removal of instruments into the patient can cause trauma and irritation to the passage of the patient, and thus it is desirable to minimize the number of times that surgical instruments need to be withdrawn and inserted/reinserted into the patient.

However, combining a shaver device with a RF wand is not straightforward. The RF plasma generated by arthroscopic RF ablation probes causes heating of the surrounding saline. In the case of suction-capable RF probes, this heated saline passes through the shaft and handle of the device, leaving via outflow tubing at the proximal end. The construction of a typical RF suction probe shaft is such that there is significant thermal insulation present between the heated saline and the outermost surface of the shaft—the surface most likely to be in contact with the skin of the patient during an arthroscopic procedure. This thermal insulation allows the suction of heated saline while ensuring the risk of a burn to the patient remains low. FIG. 1 shows this typical shaft construction. On the other hand, arthroscopic shaver probes do not typically require this thermal insulation, as the saline being drawn into the device is generally no greater than ambient temperature, and there is therefore no risk of a burn due to heated saline.

Embodiments of the present invention aim to combine these two very different devices, a shaver device and an RF suction device. Due to the geometric constraints of such a combination, this effectively results in a typical shaver construction with the additional function and components of an RF probe. The RF shaver must therefore manage the risks of heated saline.

However, it is more difficult to implement thermal insulation into the RF shaver shaft design than in previous RF suction probes. This is because the outer shaft inner diameter is taken up almost entirely by the inner shaft, which is the main suction path for the heated saline. This results in a device construction shown in FIG. 3 that does not include the air gap and polymer insulation layers present on typical RF suction probes. This reduction in thermal insulation leads to a greater risk of high outer shaft temperatures, which in turn leads to risk of patient burn at the point of skin-contact.

The risk is raised further by the inclusion of a flow valve within the device handpiece. Since high temperatures have been shown to be more likely when utilising low saline flow rates, a closed or slightly-open flow valve increases the likelihood of high shaft temperatures.

Blockage of the device, i.e. when debris blocks the suction path of the instrument, a common problem in arthroscopic procedures, also presents further risk, as a sudden ceasing of saline flow has been shown to quickly increase shaft temperatures.

### SUMMARY OF THE DISCLOSURE

The present disclosure addresses the above problem, by providing a temperature sensing component within the electrosurgical instrument construction. In this manner, temperatures of the instrument and/or the surgical site of the patient may be monitored to ensure that the patient is not burned by high temperatures internal or external to the instrument. This temperature sensor may be configured to detect temperatures internal or external to the instrument, for example, the temperature sensor may detect a temperature of the outer shaft of the instrument or a temperature of the surgical site. The temperature reading is then transmitted to the handpiece and/or RF generator, which would monitor the temperature readings and/or trends and act according to programmed responses. For example, if a temperature of the outer shaft of the instrument rises above a threshold, the RF energy provided by the generator may be switched off to prevent a further temperature rise.

Embodiments of the present invention provide a temperature sensing component able to monitor temperatures of the electrosurgical instrument and/or the surgical site of the patient. By monitoring these temperatures, action can be taken to ensure that the temperatures do not reach a dangerous level which may result in burns to the patient. The temperature sensing component feeds temperature data to a monitoring module which is able to process the data and act accordingly.

In view of the above, from a first aspect, the present disclosure relates to a temperature sensing system for an electrosurgical instrument. The temperature sensing system comprises at least one temperature sensor arranged in use to detect one or more temperatures at one or more measuring points on the electrosurgical instrument and/or on a surgical site of a patient. The temperature sensing system also comprises a monitoring module arranged in use to receive one or more readings of the one or more temperatures, process the one or more readings and send a signal in dependence on a result of said processing.

Several advantages are obtained from embodiments according to the above described aspect. For example, by monitoring temperatures of the electrosurgical instrument and/or surgical site, action can be taken to ensure that the temperatures do not reach a dangerous level which may result in burns to the patient. Such temperature monitoring may be continuous or discrete. For example, if the monitoring is discrete, the temperature sensor may detect the one or more temperatures at regular time intervals, e.g. every second, every five seconds, every ten seconds, every 30 seconds, every minute, etc.

In some embodiments, the signal prompts one or more programmed responses.

In some embodiments, the one or more programmed responses are designed to (i) prevent an increase in the one or more temperatures; and/or (ii) result in a decrease in the one or more temperatures.

In some embodiments, the one or more programmed responses comprise one or more of the following:
(i) warning a user of the electrosurgical instrument that the one or more readings are above a predetermined threshold, this allows the user to take appropriate action once they are aware of the problem;
(ii) warning the user of a blockage event within the electrosurgical instrument, blockage events commonly involve tissue/debris becoming stuck in the suction path, which can result in high temperatures;
(iii) prompting the user to increase a flow rate of a suction pump connected to the electrosurgical instrument, as slow flow rates are likely to increase temperatures;
(iv) prompting the user to open a flow valve located on a handpiece of the electrosurgical instrument, as a closed or partially closed flow valve is likely to increase temperatures;
(v) opening the flow valve, this can be done mechanically without user intervention where necessary;
(vi) sending a signal to the suction pump requesting an increased flow rate, as increased flow rates may reduce device temperatures;
(vii) reducing power of the RF output of an RF electrosurgical generator connected to the electrosurgical instrument, reducing the RF power may reduce the temperature of the RF heated saline, therefore reducing the temperature of the device;
(viii) modulating an RF waveform of the RF output to reduce average RF output power; and
(ix) switching off the RF output, this will stop the saline from being further heated by the RF energy.

In some embodiments, the at least one temperature sensor is a discrete component independent of other components within the electrosurgical instrument.

From a second aspect, the present disclosure relates to an electrosurgical instrument comprising: an end effector; an operative shaft having RF electrical connections and drive componentry for the end effector; and a temperature sensing system as described above in relation to the first aspect.

In some embodiments, the at least one temperature sensor is located within a subassembly which comprises one or more of the RF electrical connections. For example, the subassembly may include the RF active wire and/or the RF return wire.

In some embodiments, the subassembly is a laminated strip extending from a distal end of the operative shaft to a proximal end of the operative shaft. In such an embodiment, each conductive path of the RF electrical connections may split off from the strip and connect to its respective printed circuit board (PCB) or connection location.

In some embodiments, the at least one temperature sensor is located within a flexi-PCB strip which extends from a proximal end of the electrosurgical instrument to a region where the temperature sensor is located. The proximal end of the electrosurgical instrument may be a handpiece of the electrosurgical instrument.

In some embodiments, the flexi-PCB strip is connected to a rigid PCB located at the proximal end of the electrosurgical instrument. The rigid PCB may be located within the hub of the electrosurgical instrument (located in the handpiece). The connections between the flexi-PCB strip and the rigid PCB could be via soldering, crimping or a board-mounted connector.

In some embodiments, the flexi-PCB strip is integrated with the rigid PCB, thereby forming a discrete flexi-rigid PCB component.

In some embodiments, one of the one or more measuring points is located on the operative shaft of the electrosurgical instrument. This is advantageous as this is where high temperatures are likely to occur due to the RF heated saline being suctioned away from the end effector via the suction path within the operative shaft.

In some embodiments, the at least one temperature sensor is located at one or more of: (a) within the end effector; (b) on the operative shaft; or (c) within a hub located at a proximal end of the electrosurgical instrument.

In some embodiments, the at least one temperature sensor comprises at least a first temperature sensor and a second temperature sensor, the first temperature sensor arranged in use to detect a first temperature at one or more measuring points on the electrosurgical instrument and the second temperature sensor arranged in use to detect a second temperature at one or more measuring points on the surgical site.

In some embodiments, the electrosurgical instrument further comprises a rotary shaver arrangement located within the end effector, the rotary shaver arrangement being operably connected to the drive componentry to drive the rotary shaver to operate in use.

In some embodiments, the electrosurgical instrument further comprises an active electrode arrangement located within the end effector, the active electrode arrangement being operably connected to the RF electrical connections.

The temperature sensor is particularly useful for electrosurgical instruments which combine an RF suction probe and a rotary shaver device, as explained in the background section. This is due to the lack of space for thermal insulation which can result in high shaft temperatures which, without careful monitoring, could burn a patient in use.

From a third aspect, the present disclosure relates to an electrosurgical system, comprising: an RF electrosurgical generator; a suction pump; and an electrosurgical instrument as described above in relation to the second aspect, the arrangement being such that in use the RF electrosurgical generator supplies an RF output comprising a coagulation or ablation signal via the RF electrical connections to the active electrode arrangement, and the suction pump supplies suction via a suction path connecting one or more suction apertures located within the end effector to the suction pump.

In some embodiments, one of the one or more suction apertures is located within the rotary shaver arrangement. A vacuum may be applied through the inner shaft such that the bodily material that is to be cut, shaved, etc. is drawn into the windows of the inner and outer shafts by the suction when those windows become aligned, thereby facilitating the cutting, shaving, etc. of the tissue, which then travels through the inner tube due to the suction. The suction aperture may be the cutting windows.

In some embodiments, one of the one or more suction apertures is located within the active electrode arrangement. This allows the surgeon to apply suction to the surgical site without performing cutting with the surgical instrument.

In some embodiments, the monitoring module is located within the electrosurgical instrument or the RF electrosurgical generator.

From a further aspect, an electrosurgical instrument is provided with RF, mechanical shaver, and suction capabilities. The electrosurgical instrument comprises an end effector and an operative shaft having RF electrical connections and drive componentry for the end effector. The end effector comprises a rotary shaver arrangement, the rotary shaver arrangement being operably connected to the drive componentry to drive the rotary shaver to operate in use. The end effector further comprises an active electrode arrangement, the active electrode arrangement being operably connected to the RF electrical connections. The active electrode arrangement may comprise one or more suction apertures arranged to transport debris and/or fluid away from the surgical site via a suction path extending from the suction aperture through a lumen within the operative shaft.

From a yet further aspect, an electrosurgical system is provided comprising an RF electrosurgical generator, a suction pump and an electrosurgical instrument as described immediately above. The arrangement is such that in use the RF electrosurgical generator supplies an RF output comprising a coagulation or ablation signal via the RF electrical connections to the active electrode arrangement, and the suction pump supplies suction via the suction path connecting the one or more suction apertures located within the end effector to the suction pump.

When the electrosurgical instrument and/or electrosurgical system described immediately above are in use, the RF energy applied by the electrode may result in RF heated saline. This heated saline is then suctioned away from the surgical site through the suction aperture within the electrode. The heated saline therefore travels down the suction path through the lumen of the inner shaft. Due to a lack of space for thermal insulation between the lumen and the outer shaft, this hot saline may heat the outer shaft of the instrument to high temperatures. Such high temperatures could potentially burn the patient if not monitored and controlled accordingly. Therefore, such an instrument would be improved by the integration of a temperature sensing system as described in the first three aspects.

### BRIEF DESCRIPTION OF THE DRAWINGS

Embodiments of the invention will now be further described by way of example only and with reference to the accompanying drawings, wherein:

FIG. 1 is a CAD image showing a typical RF suction probe shaft construction (VAPR Tripolar 90);

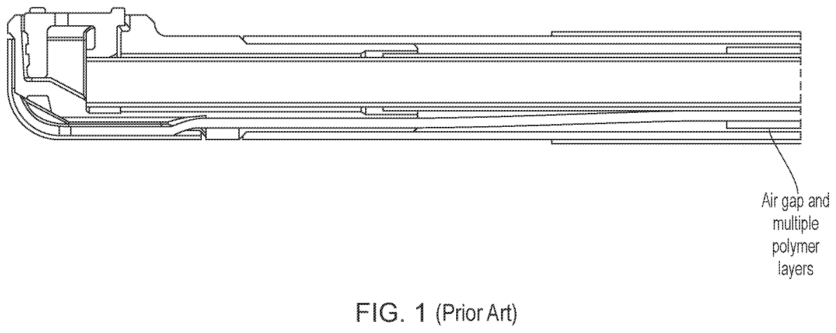

FIG. 2 is a schematic diagram of an electrosurgical system including an electrosurgical instrument;

FIG. 3 is a CAD image showing the shaft construction of an RF shaver design concept;

FIG. 4 is a CAD image illustrating a first embodiment of the present invention;

FIG. 5 is a CAD image illustrating the first embodiment of the present invention;

FIG. 6 is a CAD image illustrating a second embodiment of the present invention;

FIG. 7 is a CAD image illustrating the second embodiment of the present invention;

FIG. 8 is a CAD image illustrating a third embodiment of the present invention; and

FIG. 9 is a CAD image illustrating the third embodiment of the present invention.

### DESCRIPTION OF THE EMBODIMENTS

Embodiments of the present invention provide a temperature sensing system for an electrosurgical instrument able to detect temperatures internal and/or external to the electrosurgical instrument. The temperature sensing system comprises at least one temperature sensor and a monitoring module. Temperatures detected by the temperature sensor are processed by the monitoring module which prompts action to reduce temperatures and/or prevent further rising of temperatures where appropriate, for example, switching off the RF energy provided by the generator and/or opening a flow valve in the handpiece of the instrument. The temperature sensing system is particularly useful for electrosurgical instruments which combine rotary shaver arrangements and RF electrode arrangements, where suction is used to remove RF heated saline from the surgical site. Without monitoring the temperature of the electrosurgical instrument and/or the surgical site, there is a risk of burning the patient if the RF heated saline becomes too hot as the electrosurgical instrument may not be adequately insulated.

In more detail, one example of an electrosurgical instrument within which the temperature sensing system may be advantageously integrated is a dual-sided RF shaver with suction capabilities. Such an instrument has a rotary arrangement of two concentric cylindrical shafts, an inner shaft and an outer shaft. The inner shaft rotates relative to the outer shaft. Both the outer and inner shafts have a cutting window at their distal ends. Suction is applied along a suction path which extends from the proximal end of the instrument through the lumen of the inner shaft and through the cutting window. The suction draws tissue to be cut into the cutting window, where it is severed by the rotating inner shaft. The inner shaft may have serrated edges along its cutting window. The tissue is then suctioned into the lumen and taken away from the surgical site. In addition to the shaver capability, the instrument also has RF capabilities by virtue of an RF electrode mounted on the opposite side of the operative shaft to the outer shaft's distal cutting window. The RF electrode may be used to cut, coagulate, desiccate or fulgurate tissue. Within the electrode is a suction aperture which is also connected to the lumen of the inner shaft. A suction path extends from the lumen through the suction aperture in the electrode. This suction path is an alternative to the suction path which extends through the cutting window of the shaver side. When the surgeon wishes to apply suction via the suction aperture on the RF side, the cutting window of the shaver side is closed by rotating the inner shaft such that the cutting windows of the inner and outer shaft are misaligned. The RF energy applied by the electrode may result in RF heated plasma at the RF active tip site. Heated saline is then suctioned away from the surgical site through the suction aperture within the electrode. The heated saline therefore travels down the suction path through the lumen of the inner shaft. Due to a lack of space for thermal insulation between the lumen and the outer shaft, this hot saline may heat the outer shaft of the instrument to high temperatures. Such high temperatures could potentially burn the patient if not monitored and controlled accordingly.

Temperatures of the outer shaft (or elsewhere on the electrosurgical instrument and/or the patient) are monitored by embodiments of the present invention using a temperature sensing system. The temperature sensing system comprises a temperature sensor and a monitoring module. The temperature sensor may be mounted on the outer shaft and may have wiring connected (directly or indirectly) to the monitoring module. The monitoring module may be positioned within the handpiece of the electrosurgical instrument, or may be external to the electrosurgical instrument, for example, within the generator of the wider electrosurgical system. In use, the temperature sensor detects temperatures of its surroundings (e.g. the outer shaft) and transmits data relating to these temperatures (i.e. temperature data) to the monitoring module. The monitoring module then processes the temperature data, and if it predicts and/or detects that the temperatures are rising to potentially dangerous levels (i.e. above a predetermined threshold), the monitoring module may send signals with the aim of reducing the temperatures or at least preventing further temperature increases. Such signals may include warning a user of the high temperature, thereby prompting them to take appropriate action (e.g. opening a flow valve, clearing a blockage of the suction path, increasing the flow rate of the suction, reducing power of the RF output, and/or switching off the RF output). Alternatively, such signals may cause action to be taken without the user's direct involvement (e.g. automatically opening a flow valve, clearing a blockage of the suction path, increasing the flow rate of the suction, reducing power of the RF output, and/or switching off the RF output).

### The Electrosurgical System

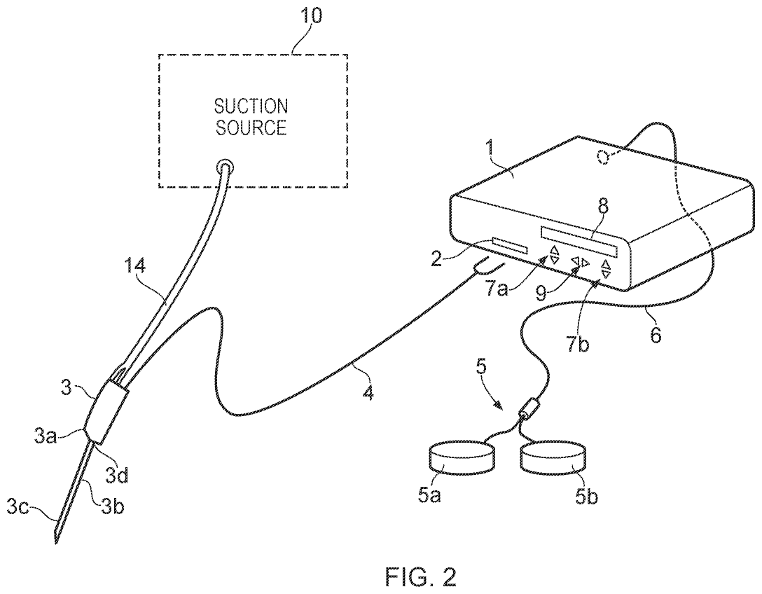

Referring to the drawings, FIG. 2 shows electrosurgical system including an electrosurgical generator 1 having an output socket 2 providing an RF output, via a connection cord 4, for an electrosurgical instrument 3. The instrument 3 has suction tubes 14 which are connected to an irrigation fluid and suction pump 10. Activation of the generator 1 may be performed from the instrument 3 via a handswitch (not shown) on the instrument 3, or by means of a footswitch unit 5 connected separately to the rear of the generator 1 by a footswitch connection cord 6. In the illustrated embodiment, the footswitch unit 5 has two footswitches 5a and 5b for selecting a coagulation mode or a cutting or vaporisation (ablation) mode of the generator 1 respectively. The generator front panel has push buttons 7a and 7b for respectively setting ablation (cutting) or coagulation power levels, which are indicated in a display 8. Push buttons 9 are provided as an alternative means for selection between the ablation (cutting) and coagulation modes.

### The Electrosurgical Instrument

The instrument 3 includes a proximal handle portion 3a, a hollow shaft 3b extending in a distal direction away from the proximal handle portion, and a distal end effector assembly 3c at the distal end of the shaft. A power connection cord 4 connects the instrument to the RF generator 1. The instrument may further be provided with activation buttons (not shown), to allow the surgeon operator to activate either the mechanical cutting function of the end effector, or the electrosurgical functions of the end effector, which typically comprise coagulation or ablation.

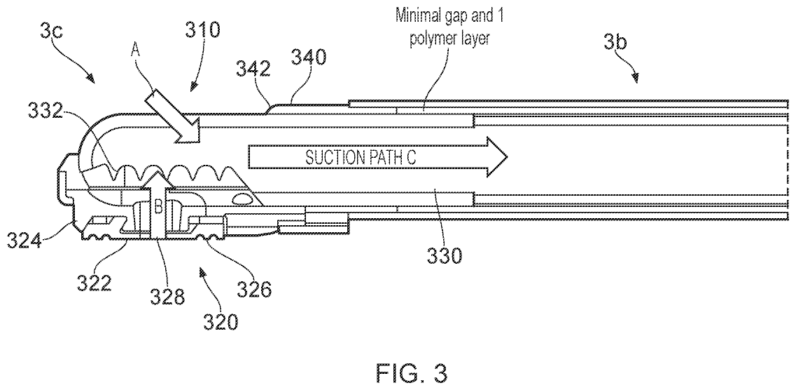

FIG. 3 shows the distal end effector assembly 3c in more detail. The distal end effector 3c has two sides to it, the shaver side 310 and the RF side 320.

The inner shaft 330 is co-axially disposed within an outer shaft 340. The outer shaft 340 has a larger diameter than the inner shaft 330. The inner shaft 330 is a tubular body having a proximal end and a distal end, with cutting window 332 disposed at a side of its distal end. The outer shaft 340 is also a tubular body having a proximal end and a distal end, with cutting window 342 disposed at a side of its distal end. The inner shaft 330 is rotatably disposed inside of the outer shaft 340 such that the surgical instrument 3 cuts tissue by rotating the inner shaft 330 within the outer shaft 340 while a vacuum is applied through the lumen of the inner shaft 330 to draw the tissue into the cutting windows 332 and 342 and sever the tissue by rotation of the inner shaft.

The RF side 320 of the electrosurgical instrument 3 comprises an electrode assembly comprising an active electrode for tissue treatment (“active tip”) 322 received in a ceramic insulator 324. The active tip 322 is provided with projections 326 to concentrate the electric field at those locations. The projections 326 also serve to create a small separation between the planar surface of the active electrode 322 and the tissue to be treated. This allows conductive fluid to circulate over the planar surface, and avoids overheating of the electrode or the tissue. The active tip 322 of the instrument is provided with a suction aperture 328, which is the opening to a lumen within an inner shaft 330.

In more detail, when the RF side 320 is to be used as a suction tool by applying a vacuum through the lumen within the inner shaft 330, the inner shaft 330 (which acts as a cutting blade) is stopped from rotating and the cutting windows 332 and 342 are misaligned with each other, i.e. closing the cutting windows, (as is the case in FIG. 3) so that the vacuum is applied through the suction path connecting the suction aperture 328 to the suction pump 10 via the lumen (i.e. the suction path defined by arrows B and C) to transport fluids to and from the active tip 322.

In contrast, when the shaver side 310 is in use for a cutting operation, suction flows via the suction path defined by arrows A and C, i.e. through the cutting windows to the lumen.

The inner and outer shafts 330 and 340 are made from a sterilisable material. For example, the sterilisable material may be a metal such as stainless steel.

### Temperature Sensing Component

Embodiments of the present invention provide a temperature sensing system comprising a temperature sensing component 402 within the construction of the electrosurgical device 3 and a monitoring module. This temperature sensor 402 is configured to monitor temperatures internal and/or external to the instrument 3. For example, the temperature sensor 402 may monitor the temperature of the outer shaft 340, and/or the temperature of the surgical site of a patient. The temperature readings would be transmitted to the monitoring module, which would monitor the temperature readings or trends and act according to the programmed responses. The monitoring module may be located in the handpiece 3a and/or the RF generator 1.

These programmed responses may include one or more of the following:
(i) warning the user that the outer shaft 340 temperature is too high;
(ii) warning the user of a blockage event, e.g. when the suction path is blocked by debris;
(iii) prompting the user to increase a flow rate of the suction pump 10;
(iv) prompting the user to open a flow valve located within the handpiece 3a;
(v) forcing the handpiece flow valve open by way of a mechanism;
(vi) sending a signal to the connected suction pump 10 requesting an increased flow rate;
(vii) reducing the power of the RF output produced by the generator 1 to reduce saline temperature;
(viii) modulating the RF waveform to reduce average RF output power produced by the generator 1; and/or
(ix) switching off the RF output from the generator 1.

The temperature sensor 402 itself could be located on any point of the instrument 3. For example, within the distal assembly 3c of the instrument, half-way along the shaft 3b, or within the instrument hub 3a.

In a two-piece design, in which there would be a disposable instrument (3b and 3c) and reusable handpiece 3a, the temperature sensor 402 would be connected to the instrument hub 3a, the component or subassembly that electrically connects the disposable device to the reusable handpiece 3a. The monitoring module which processes the temperature data and prompts actions (e.g. reduce or cut off RF output power) where necessary to reduce temperatures and/or stop the rise of temperatures may be located in the handpiece 3a and/or in the generator 1. If the monitoring module is located in the generator 1, the handpiece 3a would contain components to transfer the temperature data to the generator 1.

In a one-piece design, in which the entire device 3 is disposable, the readings taken by the temperature sensor 402 may be transferred through the device 3 and its included cable 4 to the generator 1, where the data would be processed by a monitoring module. Alternatively, the data may be processed within the disposable device 3 itself, e.g. the monitoring module may be located within the handpiece 3a, and the device 3 would respond accordingly (e.g. reduce or cut off RF output power).

In certain scenarios and designs, the temperature sensor could also be used to monitor changes in joint temperature, i.e. temperatures external to the instrument 3. The construction could include one temperature sensor that monitors temperatures both internal (e.g. outer shaft of the instrument) and external (e.g. surgical site of the patient) to the instrument, or two temperature sensors that monitor internal and external temperatures independently.

The number of temperature sensors included in the design could be increased to monitor the temperature of various locations within the surgical site and/or the instrument. For example, to obtain multiple temperature readings along the length of the device shaft.

### Temperature Sensor Component Design

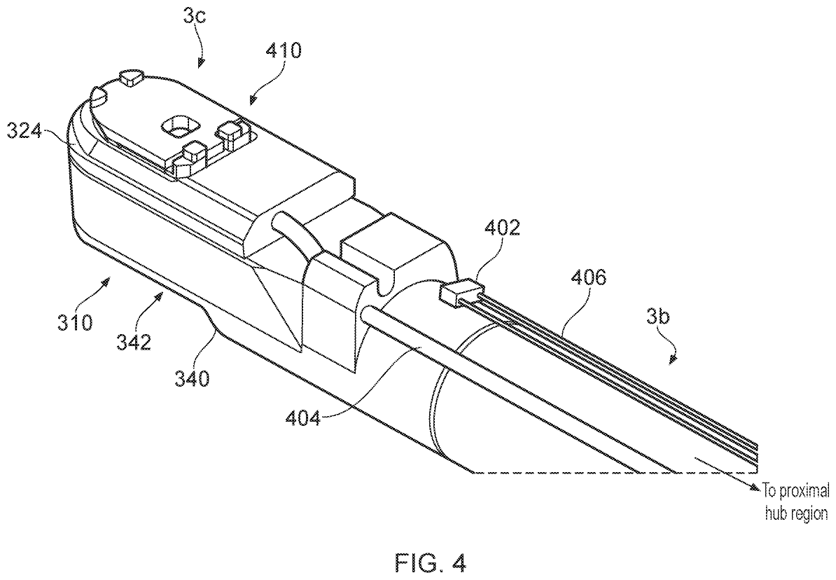

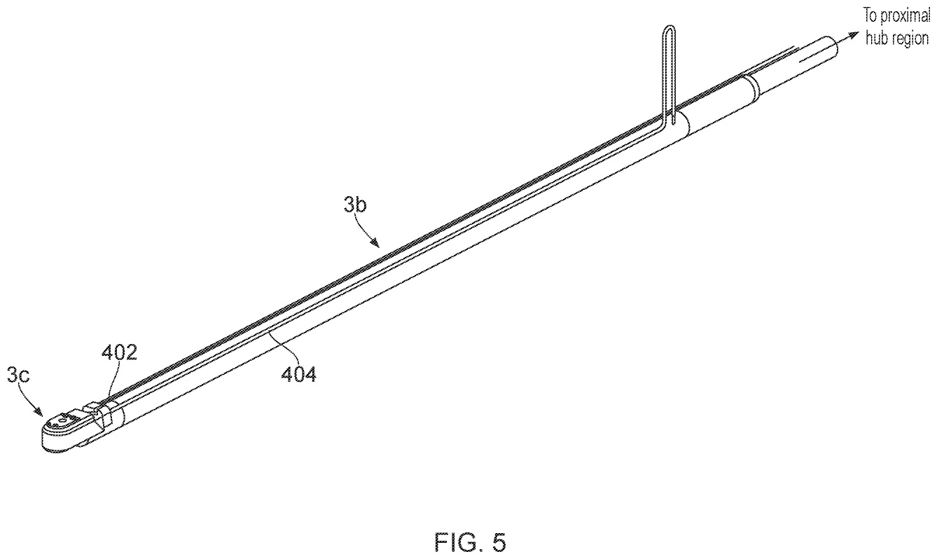

FIGS. 4 and 5 illustrate one embodiment of the present invention wherein the temperature sensor(s) 402 are discrete components independant of other components within the assembly, with wires 406 (or alternative conductive paths) extending down the shaft 3b of the instrument and into the instrument hub in the handpiece 3a. FIGS. 4 and 5 show a discrete temperature sensor 402 and RF wire component 404 running along the shaft 3b from the distal end 3c (the end effector) to the proximal hub region 3a of the instrument. The RF wire component 404 connects the end effector 3c to the proximal hub region 3a, which in turn is connected to the generator 1 by the power connection cord 4.

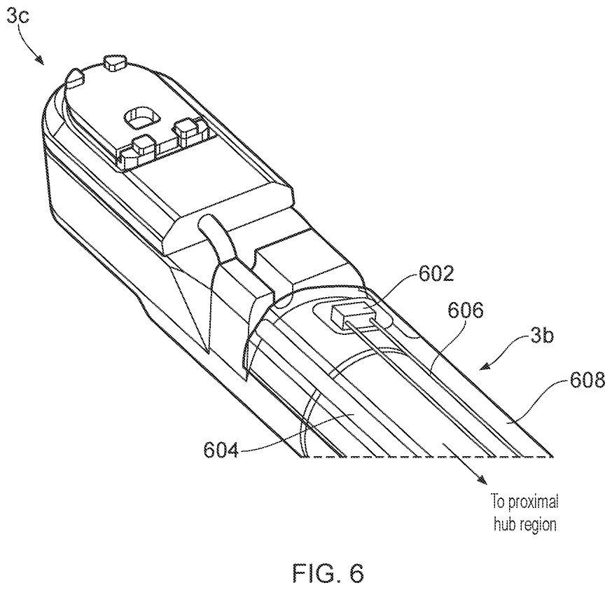

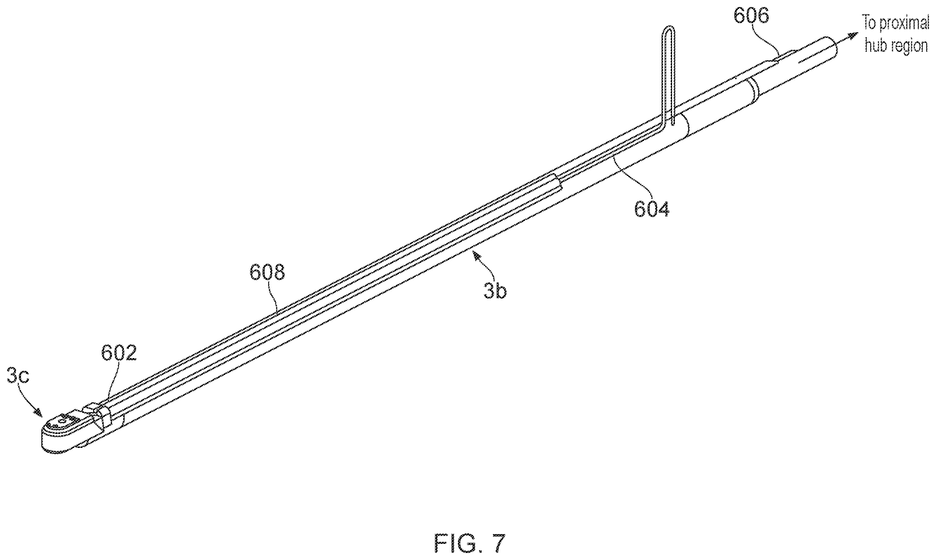

FIGS. 6 and 7 illustrate another embodiment of the temperature sensor design wherein the sensor(s) 602 could be part of a subassembly 608 that includes other conductive paths for the function of the RF instrument. For example, the RF active wire/conductive path and/or RF return wire/conductive path, e.g. 604. This subassembly 608 could, for example, house the various conductive elements and electrical components within a laminated strip 608, that would extend down the shaft 3b towards the instrument hub 3c, where each conductive path would split off and connect to its respective printed circuit board (PCB) or connection location. FIGS. 6 and 7 show a subassembly 608 with an encapsulated temperature sensor 602 and RF wire component 604 running along the shaft 3b from the distal end 3a (end effector) to the proximal hub region 3c of the instrument. The RF wire component 604 connects the end effector 3c to the proximal hub region 3a, which in turn is connected to the generator 1 by the power connection cord 4.

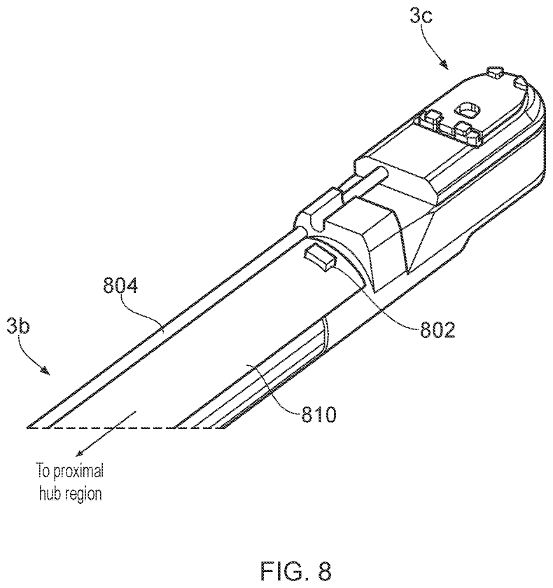

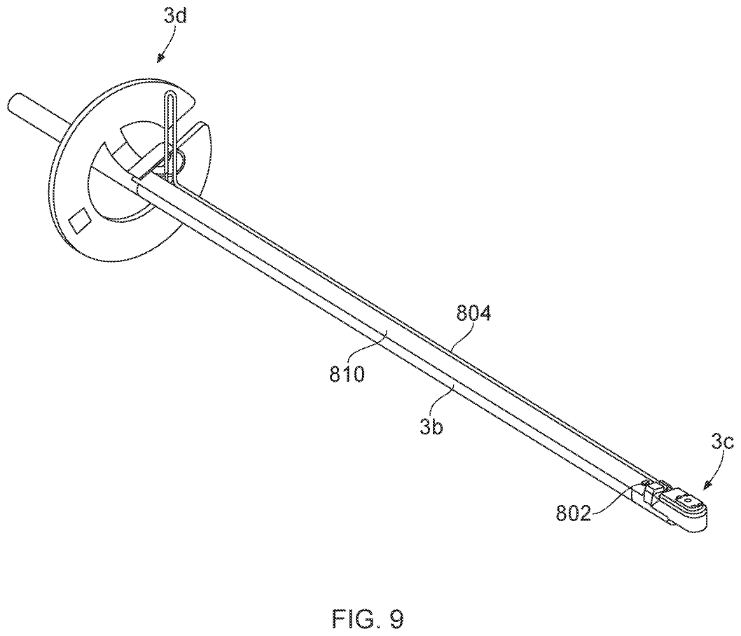

FIGS. 8 and 9 illustrate another embodiment of the temperature sensor design wherein the sensor(s) 802 could be included as part of a flexi-PCB strip 810 that would extend from the main rigid hub PCB (located in the handpiece 3a) to the location of temperature measurement on the shaft 3b (i.e., the region of the temperature sensor 802). The flexi-PCB 810 could either be integrated as part of the PCB component, forming a discrete flexi-rigid PCB component (shown in FIG. 8 below), or connected to the PCB via the methods described in the Connection sections below. This flexi-PCB component 810 could also include one or more of the RF conductive paths required for the RF function of the device. FIGS. 8 and 9 show a flexi-PCB 810 with an integrated temperature sensor 802 extending from a rigid PCB within the instrument hub and separate RF wire component 804 running along the shaft 3b from the distal end 3c to the proximal hub region 3a of the instrument. The proximal C-shape structure seen in FIG. 9 would be attached to the handpiece of the electrosurgical instrument 3a.

### Connection (Two-Piece Device Design)

In the described two-piece device design, the temperature sensor wires, e.g. 406, 606, (or other conductive paths) would need to be terminated within the instrument hub 3a. The connections could be either to a PCB via soldering, crimping or a board-mounted connector, or directly connected to the respective handpiece-mating connectors within the hub, via soldering or crimping. The handpiece-mating connectors would then transfer the data to the handpiece and then onto the generator 1 if necessary.

### Connection (One-Piece Device Design)

In the described one-piece device design, the temperature sensor wires (i.e. the thermistor wires) 406, 606 could be connected to the cable wires within the body of the disposable handle. They could also be connected to a PCB or other intermediate component within this handle if necessary, using the same methods as described for the two-piece design. Alternatively, the thermistor wires could run the length of the whole device and cable and connect directly to the generator 1.

### Use of the Technology in Other Devices and Fields

The technology described herein could also be utilised in typical RF suction probe devices. It would enable a decrease in shaft construction size, owing to the reduced need for thermal insulation within the shaft, and may therefore allow the production of low-profile RF probes that could improve tissue access in smaller joint spaces.

There is also potential for this technology to be used outside arthroscopy, in any field in which high temperatures of surgical device shafts present a risk of patient burn.

Various modifications whether by way of addition, deletion, or substitution of features may be made to above described embodiment to provide further embodiments, any and all of which are intended to be encompassed by the appended claims.

1. A temperature sensing system for an electrosurgical instrument, the temperature sensing system comprising:
at least one temperature sensor arranged in use to detect one or more temperatures at one or more measuring points on the electrosurgical instrument and/or on a surgical site of a patient; and
a monitoring module arranged in use to receive one or more readings of the one or more temperatures, process the one or more readings and send a signal in dependence on a result of said processing.

2. A temperature sensing system according to claim 1, wherein the signal prompts one or more programmed responses.

3. A temperature sensing system according to claim 2, wherein the one or more programmed responses are designed to (i) prevent an increase in the one or more temperatures; and/or (ii) result in a decrease in the one or more temperatures.

4. A temperature sensing system according to claim 3, wherein the one or more programmed responses comprise one or more of the following:
(x) warning a user of the electrosurgical instrument that the one or more readings are above a predetermined threshold;
(xi) warning the user of a blockage event within the electrosurgical instrument;
(xii) prompting the user to increase a flow rate of a suction pump connected to the electrosurgical instrument;
(xiii) prompting the user to open a flow valve located on a handpiece of the electrosurgical instrument;
(xiv) opening the flow valve;
(xv) sending a signal to the suction pump requesting an increased flow rate;
(xvi) reducing power of the RF output of an RF electrosurgical generator connected to the electrosurgical instrument;
(xvii) modulating an RF waveform of the RF output to reduce average RF output power; and
(xviii) switching off the RF output.

5. A temperature sensing system according to claim 1, wherein the at least one temperature sensor is a discrete component independent of other components within the electrosurgical instrument.

6. An electrosurgical instrument comprising:
an end effector;
an operative shaft having RF electrical connections and drive componentry for the end effector; and
a temperature sensing system comprising:
a) at least one temperature sensor arranged in use to detect one or more temperatures at one or more measuring points on the electrosurgical instrument and/or on a surgical site of a patient; and
b) a monitoring module arranged in use to receive one or more readings of the one or more temperatures, process the one or more readings and send a signal in dependence on a result of said processing.

7. An electrosurgical instrument according to claim 6, wherein the at least one temperature sensor is located within a subassembly which comprises one or more of the RF electrical connections.

8. An electrosurgical instrument according to claim 7, wherein the subassembly is a laminated strip extending from a distal end of the operative shaft to a proximal end of the operative shaft.

9. An electrosurgical instrument according to claim 6, wherein the at least one temperature sensor is located within a flexi-PCB strip which extends from a proximal end of the electrosurgical instrument to a region where the temperature sensor is located.

10. An electrosurgical instrument according to claim 9, wherein the flexi-PCB strip is connected to a rigid PCB located at the proximal end of the electrosurgical instrument.

11. An electrosurgical instrument according to claim 10, wherein the flexi-PCB strip is integrated with the rigid PCB, thereby forming a discrete flexi-rigid PCB component.

12. An electrosurgical instrument according to claim 6, wherein one of the one or more measuring points is located on the operative shaft of the electrosurgical instrument.

13. An electrosurgical instrument according to claim 6, wherein the at least one temperature sensor is located at one or more of: (a) within the end effector; (b) on the operative shaft; or (c) within a hub located at a proximal end of the electrosurgical instrument.

14. An electrosurgical instrument according to claim 6, wherein the at least one temperature sensor comprises at least a first temperature sensor and a second temperature sensor, the first temperature sensor arranged in use to detect a first temperature at one or more measuring points on the electrosurgical instrument and the second temperature sensor arranged in use to detect a second temperature at one or more measuring points on the surgical site.

15. An electrosurgical instrument according to claim 6, further comprising a rotary shaver arrangement located within the end effector, the rotary shaver arrangement being operably connected to the drive componentry to drive the rotary shaver to operate in use.

16. An electrosurgical instrument according to claim 6, further comprising an active electrode arrangement located within the end effector, the active electrode arrangement being operably connected to the RF electrical connections.

17. An electrosurgical system, comprising:
an RF electrosurgical generator;
a suction pump; and
an electrosurgical instrument, the electrosurgical instrument comprising:
i) an end effector;
ii) an operative shaft having RF electrical connections and drive componentry for the end effector; and
iii) a temperature sensing system, the temperature sensing system further comprising:
a) at least one temperature sensor arranged in use to detect one or more temperatures at one or more measuring points on the electrosurgical instrument and/or on a surgical site of a patient; and
b) a monitoring module arranged in use to receive one or more readings of the one or more temperatures, process the one or more readings and send a signal in dependence on a result of said processing;
the arrangement of the electrosurgical system being such that in use the RF electrosurgical generator supplies an RF output comprising a coagulation or ablation signal via the RF electrical connections to the active electrode arrangement, and the suction pump supplies suction via a suction path connecting one or more suction apertures located within the end effector to the suction pump.

18. An electrosurgical system according to claim 17, wherein one of the one or more suction apertures is located within the rotary shaver arrangement.

19. An electrosurgical system according to claim 17, wherein one of the one or more suction apertures is located within the active electrode arrangement.

20. An electrosurgical system according to claim 17, wherein the monitoring module is located within the electrosurgical instrument or the RF electrosurgical generator.
